(19) United States
(12) Patent Application Publication
(10) Pub. No.: US 2009/0124983 A1
(43) Pub. Date: May 14, 2009

(54) Biomedical line union device for administering pharmacological substances

(75) Inventor: Luca Ferrari, Carpi (IT)

Correspondence Address: MODIANO & ASSOCIATI, Via Meravigli, 16, MILANO, 20123, IT

(73) Assignee: LUCOMED SPA

(21) Appl. No.: 12/213,260

(22) Filed: Jun. 17, 2008

(30) Foreign Application Priority Data
Nov. 13, 2007 (IT) MO2007A000340

(51) Int. Cl. A61M 39/22; A61M 39/10

(52) U.S. Cl. 604/247; 137/798

(57) ABSTRACT

A biomedical line union device for administering pharmacological substances, comprising a union body with: at least one main duct thereinside, provided with at least one outlet associated with a conveyance line or with a closed container, and at least one branch duct, provided with at least one branching port associated with a line for administering a pharmacological substance and connected to the main duct by at least one connecting port. The device comprises at least one valve element, arranged proximate to the connecting port and movable between a closed configuration, in which it interrupts the connection between the branch duct and the main duct, and an open configuration, in which it provides the connection between the branch duct and the main duct due to an overpressure within the branch duct with respect to the pressure within the main duct, and vice versa.

The present invention relates to a union for biomedical lines, particularly for administering pharmacological substances.

### BACKGROUND OF THE INVENTION

Unions for biomedical lines are currently known which are used particularly for transfusions, infusions, hemodialysis, et cetera, and are constituted by a T-shaped or Y-shaped union body which contains a main duct and a branch duct, which is connected to the main duct and is used to administer pharmacological substances.

The main duct can be provided with an inlet and an outlet which are mutually coaxial, or can be closed at the end that lies opposite the outlet.

In the first case, the inlet and the outlet can be associated with a line for conveying a physiological fluid, generally blood, which comprises a feed tube and a discharge tube for the fluid, whereas in the second case the outlet can be associated with a closed container, such as for example a blood bag.

The branch duct, which is inclined or perpendicular with respect to the main duct, is provided with a branching port which is arranged at one of its ends and can be associated with a line, for example a syringe, for administering a pharmacological substance.

More particularly, inside the branch duct, at the branching port, there is a plug made of synthetic latex, which is retained in its seat by an interlocking cover. Such plug is adapted to be pierced with a needle of a syringe for any infusions and, by way of the elasticity of the latex, closes naturally during the extraction of the needle in order to avoid any escape of the administered pharmacological substance.

These known unions are not free from drawbacks, which include the fact that in order to administer a pharmacological substance in the fluid that flows inside the main duct or in the blood collection bag associated with the outlet, it is necessary to use a syringe provided with a needle which is adapted to pierce the latex membrane arranged at the branching port. This entails a consequent risk of injury and contamination for medical and paramedic personnel assigned to administration of the pharmacological substance.

Another drawback of known unions is that the branch duct is always connected to the main duct. This entails that any traces of pharmacological substances left on the walls of the branch duct can contaminate, even after administration has ended, the fluid that flows along the main duct or is contained in the collection bag.

Another drawback of known unions is that the cover for retaining the latex plug, being accessible from the outside, might be removed accidentally. Removal of the retention cover would allow the latex plug to escape from its seat, thus freeing the branching port and allowing the passage of unwanted contaminants through it.

### SUMMARY OF THE INVENTION

The aim of the present invention is to eliminate the above-mentioned drawbacks of the background art, by providing a union for biomedical lines which allows to administer pharmacological substances by using syringes without a needle, consequently increasing the safety of the personnel assigned to performing this operation.

Within this aim, an object of the present invention is to prevent, once administration has ended, any traces of pharmacological substances left in the branch duct from entering the main duct, contaminating the fluid that flows inside it.

Another object of the present invention is to prevent contaminants from accidentally entering the main duct.

Another object of the present invention is to provide a union for biomedical lines which is simple, relatively easy to provide in practice, safe in use, effective in operation, and has a relatively low cost.

This aim and these and other objects, which will become better apparent hereinafter, are achieved by the present union for biomedical lines, particularly for administering pharmacological substances, comprising a union body in which there is:
at least one main duct for the flow of the fluid, provided with at least one outlet which can be associated with a conveyance line or with a closed container,
at least one branch duct, which is provided with at least one branching port which can be associated with a line for administering a pharmacological substance and is connected to said main duct by means of at least one connecting port,

characterized in that it comprises at least one valve element, which is arranged proximate to said connecting port and can move between a closed configuration, in which it interrupts the connection between said branch duct and said main duct, and an open configuration, in which it provides the connection between said branch duct and said main duct due to an overpressure within said branch duct with respect to the pressure within said main duct, and vice versa.

### BRIEF DESCRIPTION OF THE DRAWINGS

Further characteristics and advantages of the present invention will become better apparent from the following detailed description of a preferred but not exclusive embodiment of a union for biomedical lines, particularly for administering pharmacological substances, illustrated by way of non-limiting example in the accompanying drawings, wherein:

FIG. 1 is a sectional side elevation view, taken along a longitudinal plane, of a union according to the invention in a first embodiment, in which the main duct is provided with an inlet and with an outlet, said union being Y-shaped;

FIG. 2 is a sectional side elevation view, taken along a longitudinal plane, of the union of FIG. 1 in the open configuration, associated with a syringe for administering a pharmacological substance;

FIG. 3 is a sectional side elevation view, taken along a longitudinal plane, of a union according to the invention, in the first embodiment, said union being T-shaped;

FIG. 4 is a sectional side elevation view, taken along a longitudinal plane, of the union of FIG. 3 in the open configuration, associated with a syringe for administering a pharmacological substance;

FIG. 5 is a sectional side elevation view, taken along a longitudinal plane, of a union according to the invention in a second embodiment, in which the main duct is closed on the opposite side of its outlet, said union being Y-shaped;

FIG. 6 is a sectional side elevation view, taken along a longitudinal plane, of the union of FIG. 5 in the open configuration, associated with a syringe for administering a pharmacological substance.

### DESCRIPTION OF THE PREFERRED EMBODIMENTS

With reference to the figures, the reference numeral 1 generally designates a union for biomedical lines, used particularly to administer pharmacological substances.

The device 1 comprises a union body 2, inside which there is a main duct 3 and a branch duct 4, which is connected to the main duct 3 for administering a pharmacological substance.

Advantageously, both the main duct 3 and the branch duct 4 are extended around respective longitudinal axes and have a substantially circular cross-section.

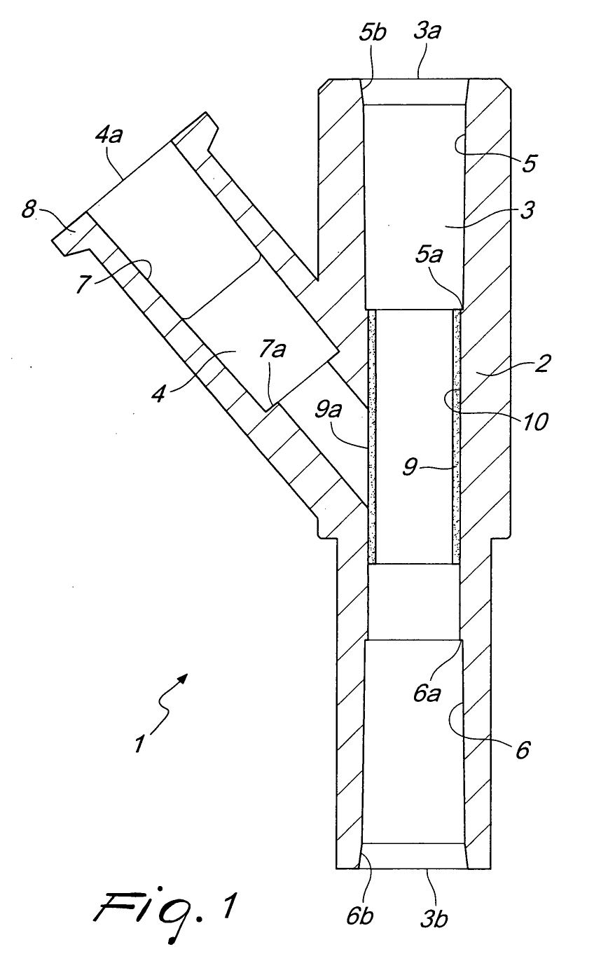

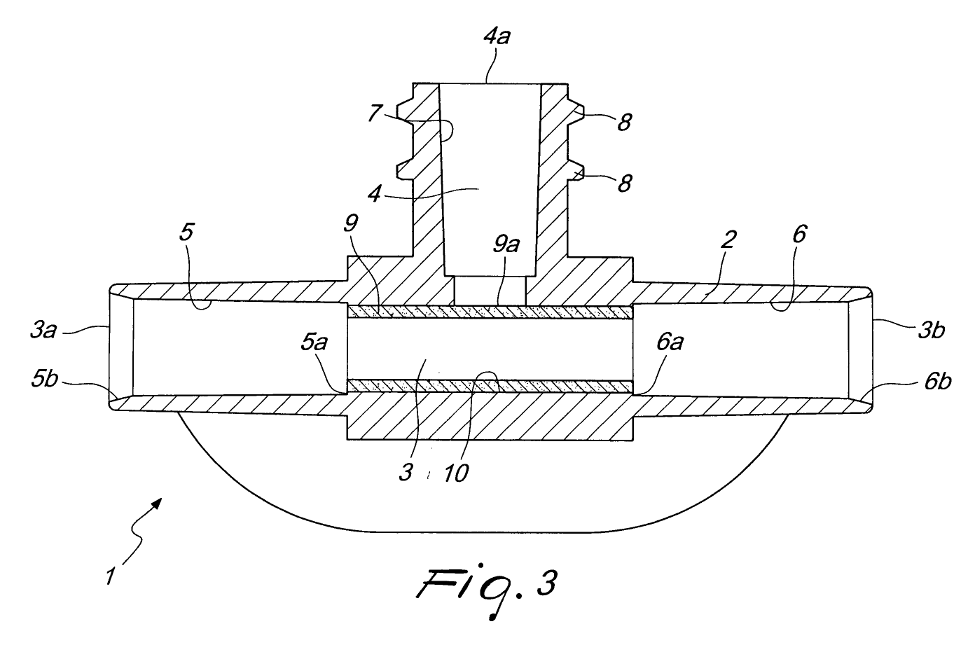

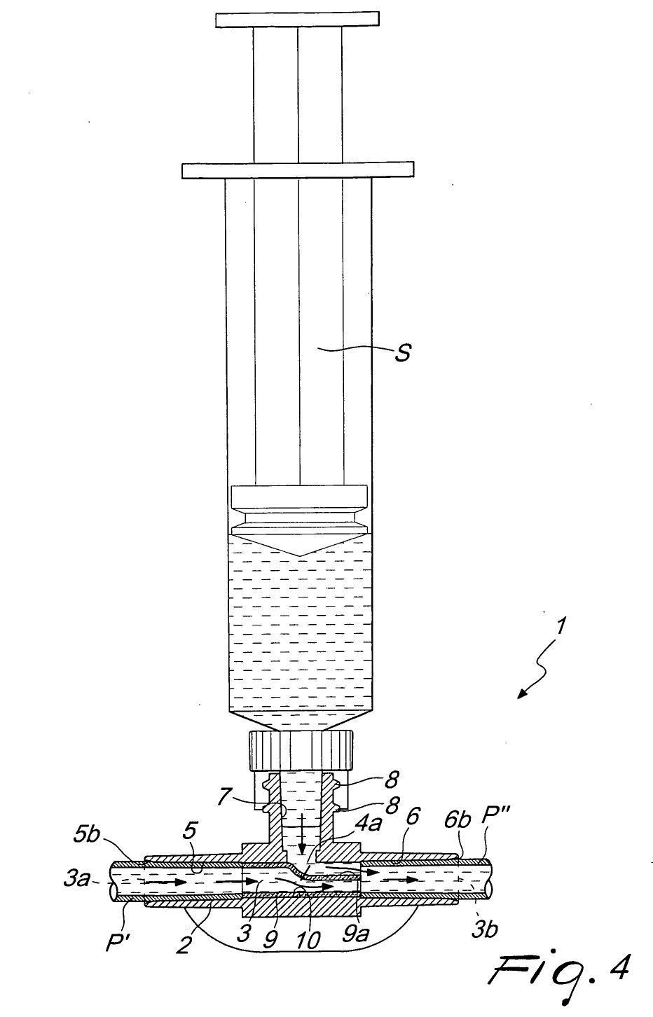

The axis of the branch duct 4 is substantially inclined or perpendicular to the axis of the main duct 3, forming in the first case a device 1 which is substantially Y-shaped, as shown in FIGS. 1, 2, 5 and 6, and T-shaped in the second case, as shown in FIGS. 3 and 4.

The main duct 3 is provided with an outlet 3b which can be associated with a conveyance line P, such as for example a line for infusion, transfusion, hemodialysis, et cetera, or with a closed container C, such as for example a blood collection bag.

In a first embodiment, shown in FIGS. 1, 2, 3 and 4, the main duct 3 is provided advantageously with at least one inlet 3a which can be associated with the conveyance line P.

In this embodiment, the main duct 3 is a duct for the flow of a physiological fluid, for example blood, and the inlet 3a and the outlet 3b can be associated respectively with a feed tube P′ and with a discharge tube P″ of the conveyance line P.

More particularly, the main duct 3 comprises a first coupling seat 5, which is formed at the inlet 3a, for accommodating the feed tube P′ of the conveyance line P, and a second coupling seat 6, which is formed at the outlet 3b, for accommodating the discharge tube P″ of the conveyance line P.

The first and second coupling seats 5 and 6 have a cross-section which increases respectively toward the inlet 3a and toward the outlet 3b and end with a shoulder, respectively 5a and 6a, for the abutment of the ends of the feed tube P′ and of the discharge tube P″ and, at the opposite end, with a flared portion, respectively 5b and 6b, which is adapted to facilitate the insertion of the feed tube P′ and of the discharge tube P″.

Conveniently, the outside diameter of the feed and discharge tubes P′ and P″ is identical to, or slightly greater than, the minimum diameter respectively of the first and second coupling seats 5 and 6, in order to provide a good seal.

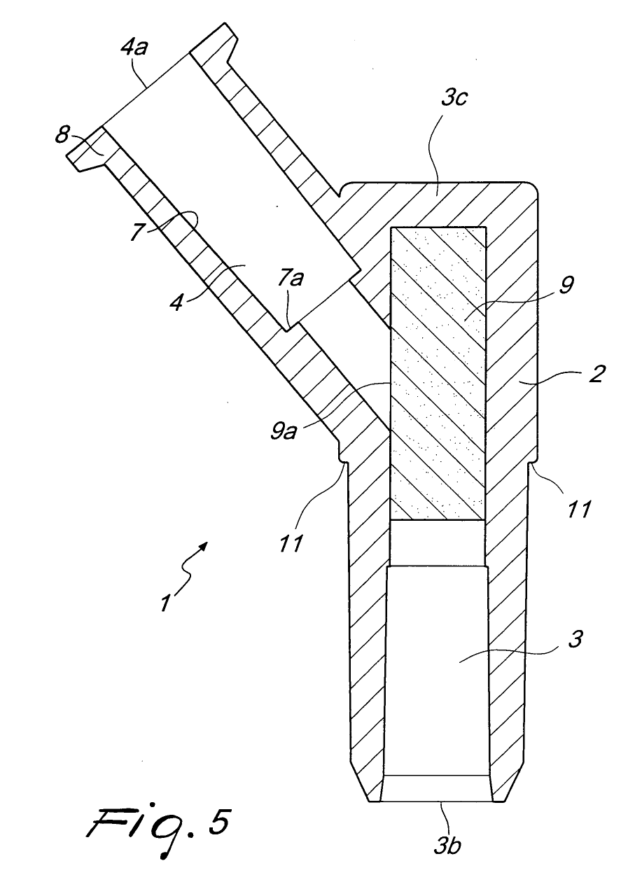

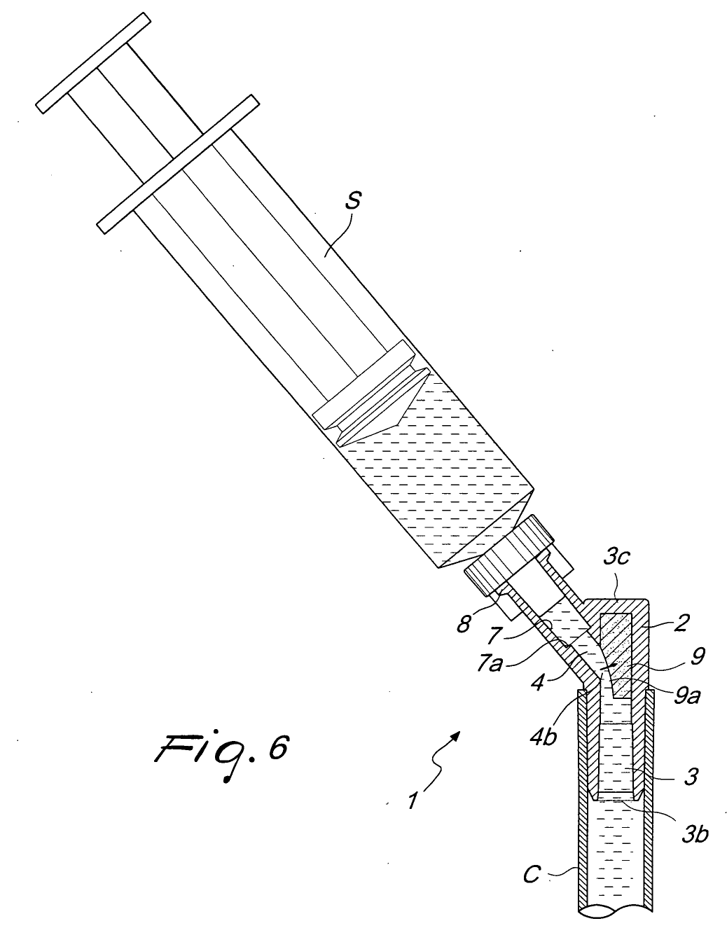

In a second embodiment, shown in FIGS. 5 and 6, the main duct 3 is closed on the opposite side of the outlet 3b by a bottom 3c which is formed by the union body 2.

In this embodiment, the outlet 3b can be associated with a blood collection bag C, the end of which can be inserted within the second coupling seat 6 or fitted externally with respect to the union body 2 so as to rest against shoulders 11 formed by such union body.

The branch duct 4 comprises a branching port 4a, which can be associated with a line S for administering a pharmacological substance, for example a syringe, and is connected to the main duct 3 by means of a connecting port 4b.

Conveniently, the branch duct 4 comprises a Luer-type receptacle 7, which has a conical shape which diverges outward and is formed at the branching port 4a to accommodate the syringe S.

More particularly, in the Y-shaped version of the union body 2, the Luer-type receptacle 7 ends with a shoulder 7a for the abutment of the end part of the syringe S. Generally, the end part of the syringe S in any case has a larger outside diameter than the smaller cross-section of the Luer-type receptacle 7, so as to ensure a good seal.

In a particular embodiment, the union body 2 comprises locking means 8, which are formed on its outer surface at the Luer-type receptacle 7, and can engage the syringe S to retain it on said union body, thus forming a Luer-Lok type receptacle 7.

According to the invention, the device 1 comprises at least one valve element 9, which is arranged proximate to the connecting port 4b and can move between a closed configuration, in which it interrupts the connection between the branch duct 4 and the main duct 3, and an open configuration, in which it provides the connection between the branch duct 4 and the main duct 3 by way of an overpressure in the branch duct 4 with respect to the pressure that is present in the main duct 3.

Advantageously, the valve element 9 is constituted by an elastically deformable tubular element, made for example of synthetic latex or silicone, which is open at its ends and is arranged so that a region 9a of its outer lateral surface lies at the connecting port 4b.

The region 9a of the tubular element 9 obstructs the connecting port 4b in the closed configuration and is deformed, for example due to an overpressure, applied by the syringe S inserted in the Luer-type receptacle 7, with respect to the pressure that is present in the main duct 3, in order to provide the open configuration, subsequently returning to the closed configuration when the overpressure ceases.

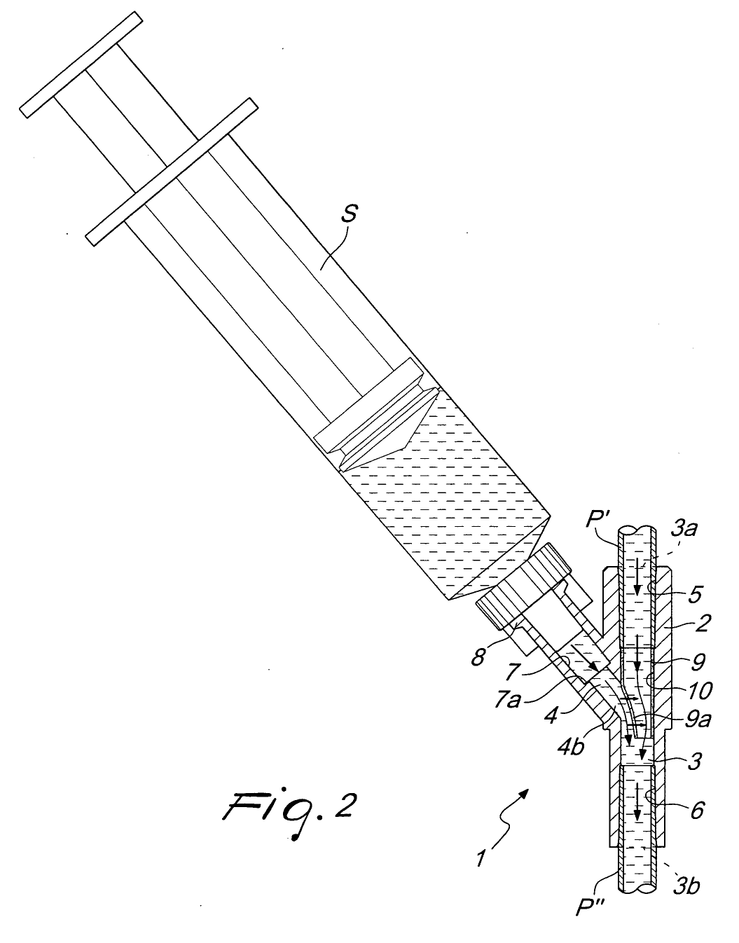

More particularly, in the open configuration shown in FIGS. 2 and 4, the region 9a is curved toward the inside of the main duct 3, consequently opening the connecting port 4b and thus allowing the passage of the pharmacological substance from the branch duct 4 toward the main duct 3.

Preferably, the tubular element 9 is arranged in a seat 10 which is formed along the main duct 3.

The tubular element 9 inserted in the seat 10 is arranged coaxially to the main duct 3.

Conveniently, the outside diameter of the tubular element 9 is slightly larger than the diameter of the seat 10, so that it remains in contact with the seat due to the radial pressure caused by the elasticity of the material.

In the first embodiment shown in FIGS. 1, 2, 3 and 4, i.e., in which the main duct 3 is provided with the inlet 3a, the seat 10 is interposed between the first coupling seat 5 and the second coupling seat 6 and the diameter of its cross-section is smaller than the minimum diameter of the cross-sections of the first and second coupling seats.

During use, i.e., when the device 1 is associated with the conveyance line P, the tubular element 9 is advantageously retained in the seat 10, in an axial direction, by shoulders which are formed by the feed tube P′ at one end and by the discharge tube P″ at the opposite end. The inside diameter of the feed tube P′ and of the discharge tube P″ is in fact slightly smaller than the outside diameter of the tubular element 9, so as to prevent its exit from the seat 10, and preferably equal to its inside diameter, in order to reduce load losses.

In the second embodiment, shown in FIGS. 5 and 6, i.e., with the main duct 3 closed on the opposite side with respect to the outlet 3b from the bottom 3c, the tubular element 9 is arranged between the bottom 3c and the outlet 3b and its end that lies opposite the one directed toward the outlet 3b rests against the bottom 3c.

The operation of the present invention is as follows.

In normal operating conditions, the main duct 3 is associated with the conveyance line P of the physiological fluid or to the blood collection bag C, while the Luer-type receptacle 7 is free and the branch duct 4 is therefore at ambient pressure.

In this condition, the tubular element 9 is in the closed configuration, thus obstructing the connecting port 4b.

In the first embodiment, shown in FIGS. 1, 2, 3 and 4, the physiological fluid enters the main duct 3 from the inlet 3 a by means of the feed tube P′, passes through the tubular element 9, and exits from the outlet 3b by way of the discharge tube P″.

In order to administer a pharmacological substance in the main duct 3, and therefore in the physiological fluid that flows inside it, the syringe S filled with the pharmacological substance to be administered is inserted in the Luer-type receptacle 7, optionally engaging it with the locking means 8.

By pressing on the plunger of the syringe S, the pressure that is present in the branch duct 4 and acts on the region 9a that closes the connecting port 4b is increased progressively.

When the value of the pressure in the branch duct 4 exceeds the value of the pressure that is present in the main duct 3 and acts on the internal lateral surface of the tubular element 9, this imbalance generates a force which acts on the region 9a and is directed toward the inside of the main duct 3.

This force deforms the tubular element 9 at the region 9a, which curves toward the inside of the main duct 3, reaching the open configuration.

The pharmacological substance that is present in the branch duct 4 can, at this point, flow out into the main duct 3, thus mixing with the physiological fluid that flows inside it.

When the plunger of the syringe S reaches the end of its stroke, and therefore the administration of the pharmacological substance ends, the pressures in the branch duct 4 and in the main duct 3 are rebalanced and consequently the force that acts on the region 9a is cancelled and such region thus returns to the closed configuration.

The region 9a is therefore deformed only in the presence of a difference in pressure between the branch duct 4 and the main duct 3, so as to prevent, in conditions of normal operation, any traces of pharmacological substances that are present in the branch duct 4 from accidentally entering the main duct 3, contaminating the fluid that flows inside it.

The operation of the device 1 in the second embodiment, shown in FIGS. 5 and 6, is substantially similar to the operation described above.

More particularly, in this embodiment, in normal operating conditions, there is no passage of physiological fluid within the main duct 3.

Indeed, the outlet 3b is associated with the blood collection bag C and the tubular element 9 is opened to perform the infusion, by means of the syringe S associated with the branching port 4a, of pharmacological substances inside the bag.

In this case also, the region 9a of the tubular element 9 is deformed due to the overpressure generated by the syringe S within the branch duct 4 with respect to the pressure that is present in the main duct 3.

The region 9a returns to the closed configuration when the overpressure caused by the syringe S ceases, again obstructing the connecting port 4b.

In practice it has been found that the described invention achieves the proposed aim and objects, and in particular the fact is stressed that it allows to administer pharmacological substances within the main duct of the union so as to mix them with the physiological fluid that flows inside it, by using syringes or the like without needles.

The device according to the invention further prevents any traces of pharmacological substances left inside the branch duct from entering the main duct accidentally, i.e., without the intention, which can be detected by means of the pressing of the syringe plunger by the assigned personnel, to administer such substance.

The invention thus conceived is susceptible of numerous modifications and variations, all of which are within the scope of the appended claims.

The term “substantially” is to be understood as an indication of the fact that the direction, shape or configuration to which it refers has the indicated properties, but for the tolerances known as usual in the pertinent field by the person skilled in the art.

All the details may further be replaced with other technically equivalent elements.

In practice, although the materials described above are preferred, the contingent dimensions and shapes may be any according to requirements without thereby abandoning the scope of the protection of the appended claims.

The disclosures in Italian Patent Application No. MO2007A000340 from which this application claims priority are incorporated herein by reference.

What is claimed is:

1. A biomedical line union device for administering pharmacological substances, comprising:
a union body;
at least one main duct, provided inside said body and having at least one outlet which is connectable with any of a conveyance line for a physiological fluid and a closed container;
at least one branch duct connected to said main duct by way of at least one connecting port, said branch duct being provided with at least one branching port that is connectable with a line for administering a pharmacological substance;
at least one valve element, which is arranged proximate to said at least one connecting port so as to be movable between a closed configuration, in which the valve element interrupts connection between said branch duct and said main duct, and an open configuration, in which the valve element provides connection between said branch duct and said main duct following to overpressure within said branch duct with respect to a pressure within said main duct, and vice versa.

2. The device of claim 1, wherein said at least one main duct is closed on an opposite side with respect to said at least one outlet.

3. The device of claim 1, wherein said at least one main duct is provided with at least one inlet, said outlet and said inlet being associable with a line for conveying a physiological fluid.

4. The device of claim 3, wherein said valve element comprises an elastically deformable tubular element, which is open at ends thereof and is arranged along said at least one main duct and has a region of an outer lateral surface thereof which is arranged at said connecting port, said region being arranged so as to obstruct said connecting port in said closed configuration and so as to deform, for opening of said connecting port, in order to provide said open configuration.

5. The device of claim 4, wherein said at least one main duct comprises a seat for said tubular element.

6. The device of claim 5, wherein said at least one main duct and said at least one branch duct lie around respective longitudinal axes thereof, the axis of said at least one branch duct being perpendicular with respect to the axis of said at least one main duct.

7. The device of claim 5, wherein said at least one main duct and said at least one branch duct lie around respective longitudinal axes thereof, the axis of said at least one branch duct being inclined with respect to the axis of said at least one main duct.

8. The device of claim 5, wherein said tubular element is inserted in said seat and is arranged coaxially to said at least one main duct.

9. The device of claim 5, wherein said at least one main duct comprises at least one first and one second coupling seats, said seats being formed, respectively, at said inlet and at said outlet to accommodate a conveyance line, said seat being interposed between said first and second coupling seats and being locked axially by shoulders formed by the conveyance line.

10. The device of claim 9, wherein said main duct has a substantially circular cross-section and said first and second coupling seats have a cross-section which increases respectively toward said inlet and toward said outlet, a diameter of said seat being smaller than a minimum diameter of said first and second coupling seats.

11. The device of claim 1, wherein said at least one branch duct comprises at least one female Luer-type receptacle which is formed at said branching port.

12. The device of claim 11, wherein said union body comprises locking means, which are formed on an outer surface thereof at said Luer-type receptacle and are suitable to engage, for retention, the administration line.
